(19) United States
(12) Patent Application Publication
(10) Pub. No.: US 2006/0155259 A1
(43) Pub. Date: Jul. 13, 2006

(54) Stomach balloon that can be inserted and removed via mouth

(76) Inventor: Alistair MacLay, New York, NY (US)

Correspondence Address: ALISTAIR MACLAY, 383 GRAND ST 1404M, NEW YORK, NY, 10002, US

(21) Appl. No.: 11/035,370

(22) Filed: Jan. 13, 2005

(51) Int. Cl. A61M 31/00

(52) U.S. Cl. 604/516; 604/909; 604/96.01; 606/192

(57) ABSTRACT

A non-inflated balloon with a valve is attached to the end of a thin flexible tube which is inserted through the patient's mouth and esophagus into their stomach. At this point the balloon is inflated with water to the desired size to fill up space in the patient's stomach. The tube is then detached from the balloon and removed, while a valve insures the balloon remains inflated to the existing size. The balloon remains alone and inflated in the patient's stomach. It takes up space in the stomach which reduces a patient's desire and ability to overeat. When the balloon needs to be removed another tube is inserted which has a claw to grab the balloon and puncture it. This deflates it and allows for it to be dragged out.

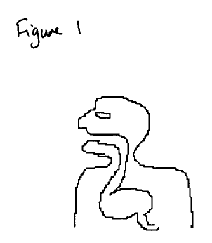

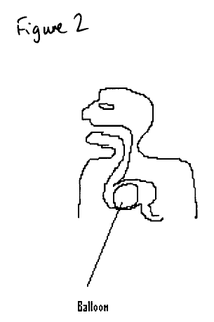

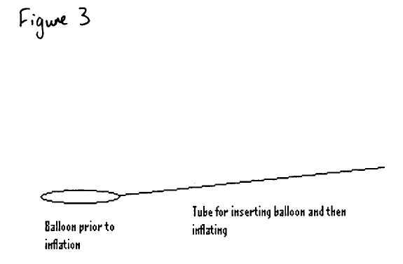

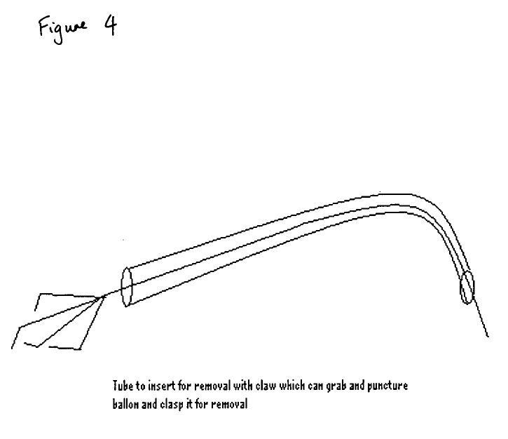

### DESCRIPTION OF THE PREFERRED EMBODIMENT OF THE INVENTION

A non-inflated balloon with a valve is attached to the end of a thin flexible tube which is inserted through the patient's mouth and esophagus into their stomach. At this point the balloon is inflated with salt water to the desired size to fill up space in the patient's stomach. The tube is then detached from the balloon and removed, while a valve insures the balloon remains inflated to the existing size. The balloon remains alone and inflated in the patient's stomach. It takes up space in the stomach which reduces a patient's desire and ability to overeat. When the balloon needs to be removed another tube is inserted which has a claw to grab the balloon and puncture it. This deflates it and allows for it to be dragged out.

The balloon may be of a size, shape and of a material which accords with the medical specialist's medical opinion of the best treatment. The medical specialist may also choose which harmless gas or liquid to fill the balloon with. The medical specialist may choose the valve to be used which will remain inside the patient as part of the balloon until removed. The medical specialist will also choose the size and type of delivery tube and removal pipe, as well as the best interface between any two of these implements.

The balloon can be molded with plastics such as Nylon, Pebax, and PET. It can be of non-spherical shape, with or without a hole through it. It can be filled with a liquid such as water or a gas such as air.

### FIGURES

1 human body pre-insertion

2 human body after insertion of stomach balloon

3 uninflated balloon attached to tube prior to insertion

4 tube with claw for puncturing, clasping and removing the balloon

### BACKGROUND AND FIELD OF INVENTION

Obesity and heart disease caused by overeating are a leading cause of death in the US. Current treatment is problematic. In the prior art a gastric band is installed which constricts the esophagus from outside. This necessitates cutting into a patient in order to insert and remove it. There is non-trivial risk of death in this procedure. Only the most immediately life threatening cases of obesity are considered for treatment this way because of the high risk. Yet millions of people overeat and thus are at risk from obesity, diet and heart-related illness.

This medical device and method provides a safer alternative to the invasive surgery which is currently used to install a gastric band or stomach staple and which has significant health risks to the patient. This safer alternative does not involve cutting through tissue or inserting and leaving in a foreign object inside the body tissue. It only places a foreign object in the stomach by way of the mouth and esophagus which areas are used to foreign objects (food) passing through them every day. The result is that more people can be treated at lower risk which will reduce their desire and ability to overeat and improve their health.

### REFERENCE

| | PAT. NO. | | Title |
|---|---|---|---|
| 1 | 6,694,982 | [T] | Gastric band |
| 2 | 6,676,674 | [T] | Gastric band |
| 3 | 5,910,149 | [T] | Non-slipping gastric band |
| 4 | 5,601,604 | [T] | Universal gastric band |
| 5 | 5,226,429 | [T] | Laparoscopic gastric band and method |
| 6 | 5,074,868 | [T] | Reversible stoma-adjustable gastric band |

What is claimed is:

1. A medical device of a stomach balloon which can be inserted through the mouth of the patient and which when inflated fills up space in a patient's stomach.

2. A medical device of a tube which attaches to balloon in claim 1

3. A medical device of a tube with a claw which can puncture the balloon in claim 1, clasp it and remove it

4. A method of treating overeating and other health and dietary issues by
A) inserting a stomach balloon as recited in claim 1
B) inflating the balloon
C) leaving the balloon in the patient's stomach
D) removing the balloon when desired

5. A medical device as recited in claim 1 which contains a therapeutic drug which is released by the device into the patient's stomach

6. A medical device which is inserted into and remains in the patient's esophagus partially blocking it

7. A medical device as recited in 1 above which is coated with a therapeutic drug

8. A medical device as recited in claim 1 which is inflated with a gas

9. A medical device as recited in claim 1 which is inflated with a liquid

10. A medical device as recited in claim 1 which is of a non-spherical shape to allow for passage of material past it

11. A medical device as recited in claim 1 which is of a non-spherical shape to allow for passage of material through it
